(19) United States
(12) Patent Application Publication
(10) Pub. No.: US 2004/0199261 A1
(43) Pub. Date: Oct. 7, 2004

(54) Surface treatment for a metal prosthesis

(75) Inventor: Eric Jones, Limerick (IE)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 10/783,245

(22) Filed: Feb. 20, 2004

(30) Foreign Application Priority Data
Feb. 24, 2003 (GB) 0304168.8

(51) Int. Cl. A61F 2/28

(52) U.S. Cl. 623/23.5; 451/38; 623/23.29

(57) ABSTRACT

A method of forming a roughened, decontaminated surface on a metal article which includes the single or multiple blasting of the surface with a chilled iron grit of appropriate roughness structure followed by acid pickling to produce a contamination free surface of substantially S_{q} 5 to 10 microns.

### BACKGROUND OF THE INVENTION

This invention relates to the surface treatment of metal and is more particularly, although not exclusively, applicable to the treatment of metal prostheses such as a femoral component.

Grits and blasting media of various types are used to roughen the surfaces of prostheses to specific extents of surface roughness to achieve optimum bone interlock features. For a cement-free femoral stem, a peaky surface is preferable to maximize primary stability, while sub-surface valleys enable new bone to interlock to provide a more long-term solution for stability. It has been demonstrated (Randelli G. Romano C., Visentin O,: Long term results of Alloclassic-Zweymuller hip prosthesis. JBJS (Br), 79 (suppl II), 238, 1997) that surfaces with an average roughness (S_{q}) of approximately 5-10 μm and peak to valley average (Sₜ) value of approximately 50-100 μm meets these requirements.

In order to produce such rough surfaces, blasting conditions and media type need to be more severe than those normally used when a matt or satin finish is required on the prosthesis. Resulting from these extreme conditions is both the embedment of the blasting media in the substrate surface and light attachment to the roughened surface. The latter can be removed relatively easily, but the embedded particles resist the cleaning methods employed and remain attached and also contribute to the overall surface roughness as proud spikes above the surface. Such contamination has been previous reported (Delaunay C. Bonnomet F. North J, Jobard D. Cazeau C. Kempf J.: Grit-blasted titanium femoral stem in cementless primary total hip arthroplasty, J. of Arthroplasty, Vol. 16, No. 1, 2001), (Bohler M., Kanz F., Schwartz B. Steffan I., Walter A. Plenk H., Knahr K.,: Adverse tissue reactions to wear particles from Co-alloy articulations, increased by alumina-blasting particle contamination from cementless Ti-based total hip implants. JBJS Vol. 84-B, No. 1, 2002), and identified as a serious concern, with the possibility of both particle detachment with consequential third body wear and inflammatory response I tissues from loose, sharp particles.

U.S. Pat. No. 5,456,723 describes a metal implant, which has a contact surface roughness of more than 20 microns to provide a good bond between the bone and the implant. This bond is significantly improved by giving the contact surface micro roughness of 2 microns or less. According to the invention this micro roughness is realized by treating a metallic body destined to become the implant with a reducing acid, which attacks the metallic surface to produce the specified micro roughness. This reducing acid may be one of a group of acids including hydrochloric acid, hydrofluoric acid, and a mixture of hydrochloric and sulphuric acids. The reducing acid is preferable made to exert its action on the implant in its boiling state. This acid treatment alone produced the results desired by effectively pitting the surface of the metal implant. Sandblasting may be used as a step preceding the reducing acid treatment.

### SUMMARY OF THE INVENTION

According to the present invention a method of forming a roughened, decontaminated surface on a medial article includes the single or multiple blasting of the surface with a chilled iron grit of appropriate roughness structure (eg 180-1190 microns) and followed by acid pickling to produce a contamination free surface of about S_{q} 5 to 10 microns.

Preferably the surface is pickled in nitric acid and in a preferred method the surface is pickled in 20% nitric acid for about 20 minutes at ambient temperature. The use of nitric acid (which is an oxidizing acid) effectively dissolves the soluble iron grit embedded or loosely held on the implant surface to provide a decontaminated surface.

The use of nitric acid effectively dissolves the iron grit embedded or loosely held on the implant surface to produce a decontaminated surface. Other dilute acids may be used that dissolve the iron grit, but do not attack the metallic implant.

Nitric acid is the preferred acid as it also passivates the surface of the metallic implant. For example, dilute hydrochloric acid (20%) can be used to remove any insoluble iron salts which may be left after treatment with nitric acid.

Ultrasonic agitation during the acid treatment process is preferable to loosen the partially solubilized iron salt from the surface of the metallic implant. It has been found that the process is particularly applicable for use with a medical article made from titanium or titanium alloy. The method may include blasting with G07 chilled iron grit at a pressure of 6.5 bar at 40 cubic meters per hour of air through a 9.5 mm nozzle and 4.8 mm air jet. The method can also include first blasting with G12 chilled iron grit at 6.5 bar air pressure through a 9 mm nozzle before blasting with the G07 grit. The blasting time in both cases can be 3 to 4 minutes with a standoff distance between 10-50 mm. In any case, at least two warm or cold-water rinses can be applied after acid treatment.

The method can be applied to a prosthesis, which has an insertion portion extending from an operative portion, the roughened decontaminated surface being formed on the insertion portion. A protective cover can be applied to the operative portion, such as a trunnion and which is used to carry the prosthesis during blasting.

Grit selection is determined by the surface hardness of the material and, as mentioned above, for an un-machined (as forged) titanium alloy it is possible to use G07 grit to obtain the required level of surface roughness.

For machined titanium alloy (the machining operation work hardens the titanium alloy surface) it is necessary, as stated above, to use two grits, a first blast with a G12 to give a rough, peaky surface. Use of a second blast with G07 can be used to cut the peaks down, if required. Stand-off distance may also be reduced in the case of harder materials, and or pressure increased, so that only one grit type needs to be used. The quality of peaky surface may be slightly reduced. If the hardness is even greater, it may be necessary to use an even rougher grade of grit to give the initial cut to the surface and then refine to the desired surface profile with a less course grit. Softer surfaces can also be accommodated with a combination of grit selection, pressure, blasting time and stand-off distance.

The acid leach does alter the roughness of the surface by a small amount. This is not, however, by attacking the surface of the alloy but by dissolving iron grit embedded in the titanium alloy surface, and which complements to the overall surface roughness.

### BRIEF DESCRIPTION OF THE DRAWINGS

The invention can be performed in various ways and one embodiment will now be described by way of example and with reference to the accompanying drawings in which:

FIG. 1 is a pictorial view of a known preferred surface feature and table showing various parameters;

FIG. 2 is a side elevation of a femoral prosthesis to which the method according to the present invention is applied;

FIG. 3 is a block diagram showing the method according to the present invention;

FIG. 4 is a block diagram showing another method according to the invention;

FIG. 5 is the EDAX profile of the Ti-alloy showing no contamination; and

FIG. 6 is the EDAX profile of Ti-alloy showing contamination by silicon, aluminum and oxygen when a known method of producing a roughened surface is used which does not include the process set forth in the present application.

### DETAILED DESCRIPTION

Some 20 years clinical history with a cementless, straight-stemmed hip has demonstrated that the typical preferred surface properties are:

Sₜ ten-point height of the surface is in the order of 50 to 100 microns.

S_{q} root mean square deviation of the surface is in the order of 5 to 10 microns.

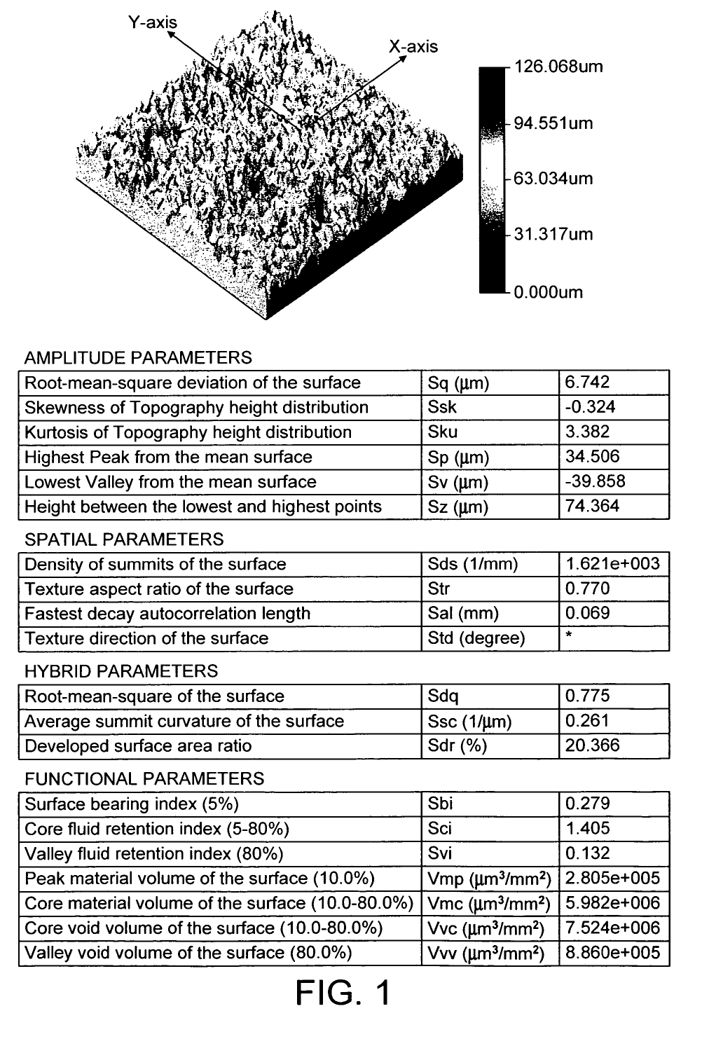

An example of the preferred surface features is shown in FIG. 1 and was measured by non-contacting surface profilometry that is a technique that is able to represent a 3-dimensional pictorial display of the surface.

In the preferred surface profile, significant quantities of asperities protrude to give good initial interlock between the stem and the bone and thus imparting early post-operative stability. There is a balance of the valleys to cater for eventual bone in-growth.

The surface roughness is generally achieved by blasting the surface to be roughened with a blasting medium such as alumina particles.

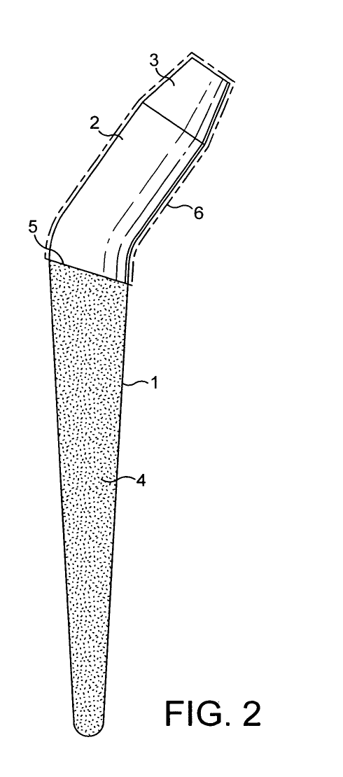

In the preferred method described, the invention is applied to a prosthesis which has an insertion portion or stem that is to be inserted into a human bone and which extends from an operative portion. FIG. 2 shows a femoral prosthesis to which the present invention can be applied. The prosthesis comprises an insertion portion in the form of stem 1 which extends from an operative portion provided by neck 2 on which is a tapered spigot or trunnion 3 to which a prosthetic head in the form of a bearing ball can be fitted in known manner. The roughened decontaminated surface indicated by reference numeral 4 is formed on stem 1.

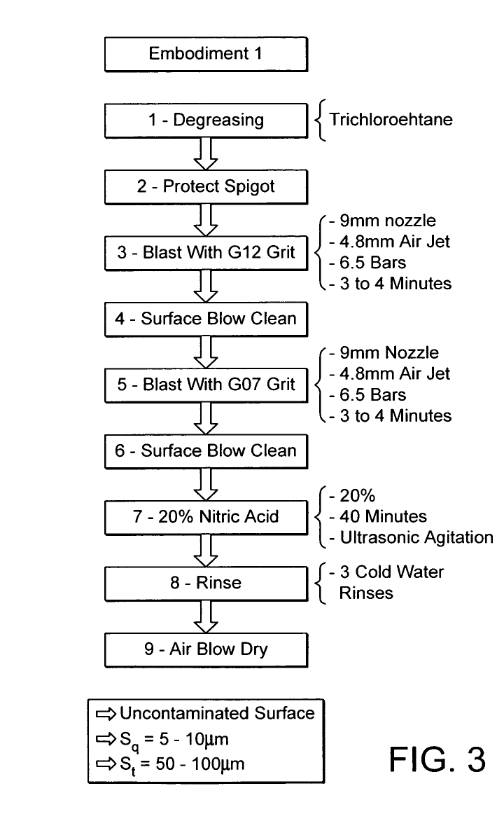

The block diagram shown in FIG. 3 illustrates the process according to the invention. Prior to treating the titanium alloy prosthesis operative portion 2 and 3 of the stem is either dipped or otherwise wrapped to provide a protective covering of polyurethane or other suitable material, which extends down to the proximal end of the part of the stem to be treated and which is indicated by reference numeral 5. Thus, all the upper part of the prosthesis above line 5 is covered by the protective coat, which is indicated by broken lines 6 in FIG. 2.

In a preferred embodiment, prior to the protective coating, however, and as shown in the flow chart of FIG. 3, the prosthesis is first degreased, for example, by using trichlorethane as indicated in Box 1. The protective coating is now applied as indicated in Box 2, and the prosthesis is now passed to blasting as indicated in Box 3. In the example being described a Guyson Multiblast six-station machine is used and the prostheses are held in position by clamps, which are attached to the protected portion provided by, for example, polyurethane covering 6. As the prosthesis is made from machined titanium alloy its outer surface has hardened so, in the method being described stem 1 is first blasted with G12 chilled iron grit for 3 minutes with a 9 mm nozzle at 6.5 bar air pressure.

The surface is then blow cleaned with air as indicated by Box 4 and is then blasted with G07 chilled iron grit, again for 3 minutes with a 9 mm nozzle at 6.5 bar pressure as shown in Box 5. The surface is again blown clear by compressed air as shown in Box 6 and in the preferred method, the prosthesis, as shown in Box 7 is placed in a bath containing 20% nitric acid for more than 20 minutes and less than 40 minutes with ultrasonic agitation.

The prosthesis is then rinsed in high pressure water as indicated in Box 8 and subsequently air blown dry (Box 9).

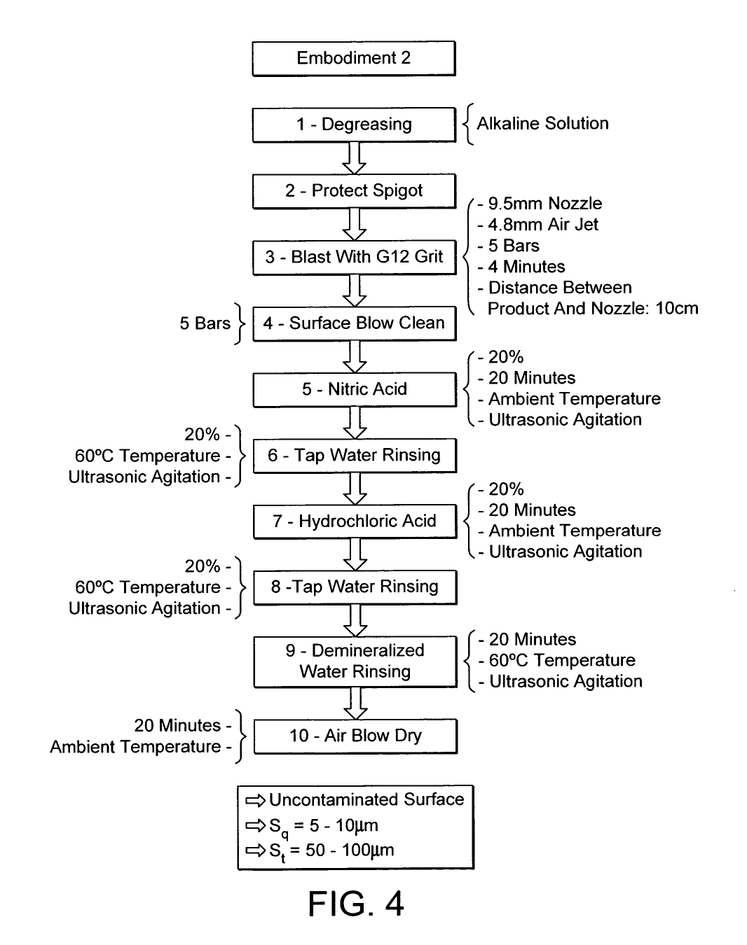

A second embodiment is shown in FIG. 4, and, as indicated Box 1, the product is first degreased using for example an alkaline solution. A protective cover is applied over the spigot, as described with reference to FIG. 3 as indicated in Box 2, and the prosthesis is now passed to the blasting station as indicated in Box 3. In the example being described a Guyson Multiblast six-station unit is again used as previously described. In this case, a single blast using the G12 chilled iron grit for 4 minutes with applied air pressure of 5 bar and 9.5 mm nozzle is employed.

The surface is then blow cleaned with air at 5 bar as indicated in Box 4 after which it is placed in a bath containing 20% nitric acid for no less than 20 minutes and no more than 40 minutes with continuous ultrasonic agitation as indicated in Box 5. As indicated in Box 6, the component is then rinsed in a bath with warmed tap water for a period of 20 minutes, again with ultrasonic agitation. The process to this point has removed all of the readily solubilized iron salts.

A further step to remove any insoluble salts utilizes pickling in a bath of 20% hydrochloric acid for more than 20 minutes and less than 40 minutes with ultrasonic agitation as described in Box 7. The prosthesis is then rinsed in a warmed tap water bath with ultrasonic agitation as indicated in Box 8. A final washing in warm demineralized water again employing ultrasonic agitation completes the cleaning process as indicated in Box 9.

The product is then air dried at ambient temperature for 20 minutes or until dry. Both embodiments described can be operated in a manual or fully automated mode, or some appropriate combination.

The specification of the chilled iron grit is shown below:

| C | Si | Co | Ni | Cr | Mo | S | P | Pb | Fe |
|---|---|---|---|---|---|---|---|---|---|
| 3.01 | 1.69 | 0.35 | 0.33 | 0.19 | 0.06 | 0.078 | 0.069 | 0.0016 | Ba- |
| | | | | | | | | | lance |

Typical chilled iron grit sizes available for use are:

G07 (180-350 microns)

G07 sieved to remove the fraction above 300 microns

G05 (150-300 microns)

G12 (250-500 microns)

G34 (710-1190 microns)

Grit selection is determined by the surface hardness of the material for the desired surface roughness. For un-machined (as forged) titanium alloy it is possible to use G07 grit only to obtain the required level of surface roughness and the stages shown in Boxes 3 and 4 will not be necessary. For the machined titanium alloy (the machining operation work hardens the titanium alloy surface) as describe above, it is necessary to use 2 grits.

A first blast with G12 gave a rough, peaky surface. A second blast with G07 cuts the peaks down. If the hardness is even greater, it may be necessary to use even rougher grit to give the initial cut into the surface.

The nitric acid leach does alter the roughness of the surface after blasting by a small amount. This is not, however, by attacking the surface of the alloy, but by dissolving the iron grit embedded in the titanium alloy surface. It has been shown that the roughness of the clean surface is unaltered by a further acid wash so indicating that the acid does not chemically attach the metal surface. The blasting time will depend upon the size of the implant and the metal, but is preferably between 3 to 4 minutes with a stand off distance of 10-50 mm.

It has been found (using EDAX probing for elemental analysis) that the surface is substantially uncontaminated apart from small quantities of zirconium and molybdenum, the nitric acid acting to dissolve the residual iron grit, and this also provides a surface, which, depending upon the resting time can be S_{q} 5-10 microns. (As determined using non-contacting surface profilometry.)

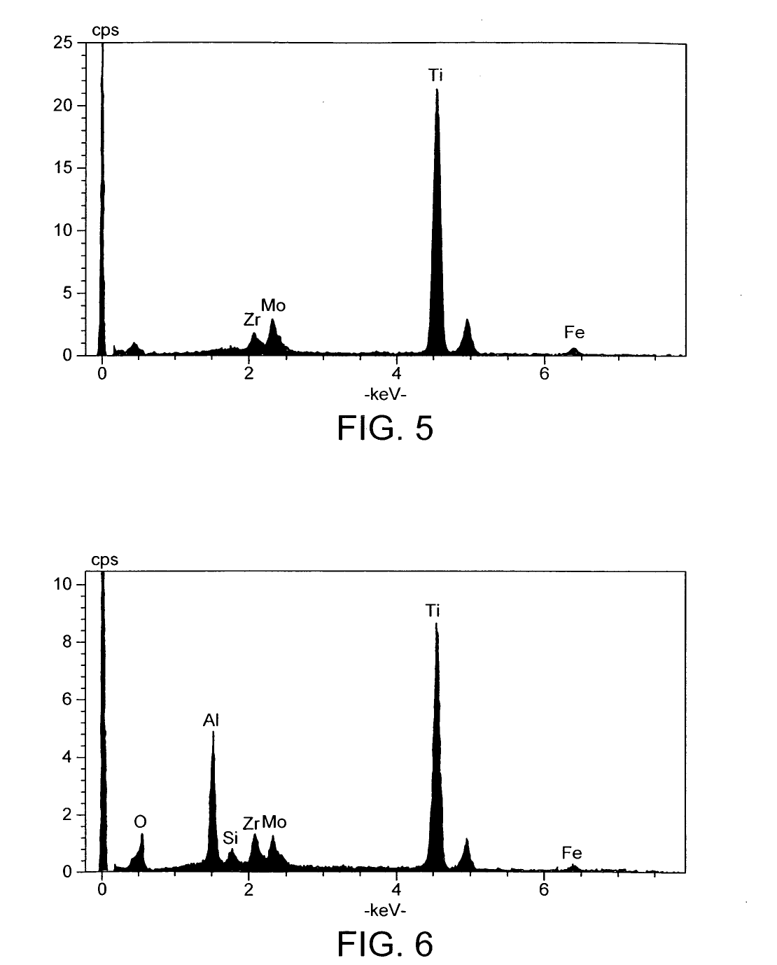

FIG. 6 is an EDAX profile similar to that shown in FIG. 5 of a Ti-alloy but showing additional contamination by oxygen, aluminum and silicon when a known method of producing a roughened surface is used which does not include the process set forth above. Comparison of FIGS. 5 and 6 clearly indicates the reduction in contamination achieved by the present invention over the previously known process.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for surface treating a metal orthopedic prosthesis having a portion for implantation in bone comprising:
blasting at least the portion for implantation on bone with a chilled iron grit and thereafter leaching out any grit on the stem surface with acid.

2. The method as set forth in claim 1 wherein said iron grit is between 180 and 1190 microns in size.

3. The method as set forth in claim 1 wherein said acid treatment is in 20% nitric acid for 20 minutes at ambient temperature.

4. The method as set forth in claim 3 which includes using 20% dilute hydrochloric acid to remove any insoluble iron salts left after treatment with the nitric acid.

5. The method as set forth in claim 4 further including the application of ultrasonic agitation during the acid treatment.

6. The method as set forth in claim 1 wherein the blasting is with a G07 chilled iron grit at a pressure of 6.5 bar at 40 cubic meters per hour of air through a 9.5 mm nozzle and a 4.8 mm air jet.

7. The method as set forth in claim 6 which includes blasting with G12 chilled iron grit at 6.5 bar air pressure through a 9 mm nozzle before blasting with G07 grit.

8. The method as set forth in claim 7 wherein the blasting time or times is 3 to 4 minutes with a stand off distance of between 10 to 50 mm.

9. The method as set forth in claim 1 wherein at least two warm or cold water rinses are applied after the acid treatment.

10. A method of forming a roughened, decontaminated surface on a metal article which includes the single or multiple blasting of the surface with a chilled iron grit of appropriate roughness structure followed by acid pickling to produce a contamination free surface of substantially S_{q} 5 to 10 μm.

11. The method as set forth in claim 10 wherein the chilled iron grit has a roughness structure of between 180 to 1190 microns.

12. The method as set forth in claim 10 wherein said acid treatment is in 20% nitric acid for 20 minutes at ambient temperature.

13. The method as set forth in claim 12 which includes using 20% dilute hydrochloric acid to remove any insoluble iron salts left after treatment with the nitric acid.

14. The method as set forth in claim 12 further including the application of ultrasonic agitation during the acid treatment.

15. The method as set forth in claim 10 wherein the blasting is with a G07 chilled iron grit at a pressure of 6.5 bar at 40 cubic meters per hour of air through a 9.5 mm nozzle and a 4.8 mm air jet.

16. The method as set forth in claim 15 which includes blasting with G12 chilled iron grit at 6.5 bar air pressure through a 9 mm nozzle before blasting with G07 grit.

17. The method as set forth in claim 16 wherein the blasting time or times is 3 to 4 minutes with a stand off distance of between 10 to 50 mm.

18. The method as set forth in claim 10 wherein at least two warm or cold water rinses are applied after the acid pickling.

20. The method as set forth in claim 10 wherein the metal article is a prosthesis which has an insertion portion extending from an operative portion, and in which the roughened decontaminated surface is formed on the insertion portion.

21. The method as set forth in claim 20 which includes applying a protective cover to the operative portion.

22. The method as set forth in claim 10 wherein the metal is a titanium alloy.

23. A prosthetic titanium or titanium alloy implant comprising a bone contacting portion having a surface with an average surface roughness S_{q} of 5-10 μm, said surface being substantially free of aluminum and silicon contamination when measured by an EDAX elemental analysis.
